SEQUENCE LISTING (8 sequences)

SEQ ID NO: 1 - DNA, 20 bp, synthetic construct
  source 1..20: mol_type = other DNA
gctgcgaagtggaaaccatc

SEQ ID NO: 2 - DNA, 22 bp, synthetic construct
  source 1..22: mol_type = other DNA
cctccttctgcacacatttgaa

SEQ ID NO: 3 - DNA, 23 bp, synthetic construct
  source 1..23: mol_type = other DNA
aataaggcgaagatcaacatggc

SEQ ID NO: 4 - DNA, 23 bp, synthetic construct
  source 1..23: mol_type = other DNA
tttgttaccaatgtccccaagag

SEQ ID NO: 5 - DNA, 21 bp, synthetic construct
  source 1..21: mol_type = other DNA
agacctgggcagattccaaac

SEQ ID NO: 6 - DNA, 20 bp, synthetic construct
  source 1..20: mol_type = other DNA
cggcaagtcttccgagtagt

SEQ ID NO: 7 - DNA, 21 bp, synthetic construct
  source 1..21: mol_type = other DNA
catgtacgttgctatccaggc

SEQ ID NO: 8 - DNA, 21 bp, synthetic construct
  source 1..21: mol_type = other DNA
ctccttaatgtcacgcacgat

(19) United States
(12) Patent Application Publication
(10) Pub. No.: US 2023/0331676 A1
(43) Pub. Date: Oct. 19, 2023

(54) NAPHTHYLUREA COMPOUND, METHODS OF PREPARATION AND USE THEREOF

(71) Applicant: Henan Radiomedical Science and Technology Co., Ltd., Kaifeng (CN)

(72) Inventors: Marvin Xuejun XU, Kaifeng (CN); Yupo YANG, Kaifeng (CN); Zhengyan YANG, Kaifeng (CN); Hongyun XU, Kaifeng (CN); Chaoqun DUAN, Kaifeng (CN); Jiawei DONG, Kaifeng (CN)

(21) Appl. No.: 18/314,102

(22) Filed: May 8, 2023

Related U.S. Application Data
(63) Continuation-in-part of application No. PCT/CN2022/092155, filed on May 11, 2022.

(30) Foreign Application Priority Data
Apr. 8, 2021 (CN) 202110376650.6
Jun. 29, 2021 (CN) 202110727896.3

(51) Int. Cl. C07D 213/40; A61P 35/02; C07D 239/26; C07D 237/08; C07D 241/12; C07D 211/26; C07D 309/04; C07D 309/06; C07D 307/14

(52) U.S. Cl. CPC C07D 213/40; A61P 35/02; C07D 239/26; C07D 237/08; C07D 241/12; C07D 211/26; C07D 309/04; C07D 309/06; C07D 307/14

(57) ABSTRACT

The disclosure provides a naphthylurea compound having a formula I. In the formula, m and n represent a number of CH₂, and are an integer from 1 to 10; k and z represent a number of CH₂, and are an integer from 0 to 6; and p represents a number of CH₂, and is 1, 2 or 3; and X is O or S.

### CROSS-REFERENCE TO RELATED APPLICATIONS

This application is a continuation-in-part of International Patent Application No. PCT/CN2022/092155 with an international filing date of May 11, 2022, designating the United States, now pending, and further claims foreign priority benefits to Chinese Patent Application No. 202110376650.6 filed Apr. 8, 2021, and to Chinese Patent Application No. 202110727896.3 filed Jun. 29, 2021. The contents of all of the aforementioned applications, including any intervening amendments thereto, are incorporated herein by reference. Inquiries from the public to applicants or assignees concerning this document or the related applications should be directed to: Matthias Scholl P. C., Attn.: Dr. Matthias Scholl Esq., 245 First Street, 18th Floor, Cambridge, MA 02142.

### INCORPORATION-BY-REFERENCE OF SEQUENCE LISTING

This application contains a sequence listing, which has been submitted electronically in XML file and is incorporated herein by reference in its entirety. The XML file, created on Mar. 15, 2023, is named ZZLK-03601-UUS.xml, and is 8,128 bytes in size.

### BACKGROUND

The disclosure relates to the field of target therapy for cancer, and more particularly, to a naphthylurea compound, methods of preparation and use thereof.

Abnormal activation of JAKs (Janus kinases)/STATs (Signal transducers and activators of scripts) signals is associated with cancer and immune-related diseases. The JAKs kinase family includes four members: JAK1, JAK2, JAK3 and Tyk2. They all contain seven domains, JH1-JH7. The JH1 domain is considered to have tyrosine kinase activity and can catalyze the phosphorylation of substrates (such as STATs).

The overexpression and constitutive activation of JAK2/STAT3 are common in many solid tumors and hematological cancers. STAT3 is a member of the STATs family and a substrate protein of JAK2, and closely related to the formation, development and malignant transformation of cancer. Under normal conditions, STAT3 exists in the cytoplasm in the form of inactive monomers. When the negative feedback regulation mechanism of JAK2 or STAT3 is abnormal or gene mutation occurs, the phosphorylation level of STAT3 continuously increases and is endogenously exited, and the STAT3 protein forms a homodimer or heterodimer with the SH2 domain of another STAT3 protein. The homodimer or heterodimer enters the nucleus, binds to specific gene promoter sequence through the DNA binding domain, thus starting the transcription of downstream genes and the expression of a series of anti-apoptotic factors such as BCL-2, BCL-XL, CyclinD1, etc.

Because many pro-proliferation, invasion and anti-apoptosis genes, such as CyclinD1, Bcl-xl, MMP9 and c-Myc, are the target genes of JAK2/STAT3 signal, in the animal tumor model or tumor cells cultured in vitro with continuously activated STAT3, the inhibition of JAK2 or STAT3 protein can effectively inhibit the growth of tumor cells or induce tumor cell apoptosis, and reduce the metastasis of tumor cells. JAK2 and STAT3 have become hot targets for tumor treatment. However, the demand of JAKs/STAT3 inhibitors in the tumor market is far from being met.

### SUMMARY

The disclosure provides a naphthylurea compound, uses of derivatives thereof in treatment of tumor, targets thereof, and an anti-tumor mechanism thereof. By some biological analysis techniques, the naphthylurea compound has been found to be effective anti-tumor agents that inhibit proliferation and development of tumor cells within liver cancer, breast cancer, lung cancer and leukemia, causing the tumor cells to be arrested in the G1/S or G2/M phase of the cell cycle and undergo apoptosis.

The objective of the disclosure is to provide a naphthylurea compound, methods of preparation and use thereof.

The naphthylurea compound have the following formula I:

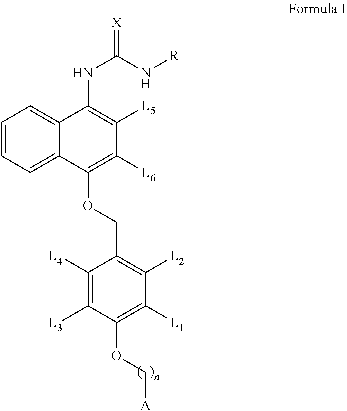

R represents

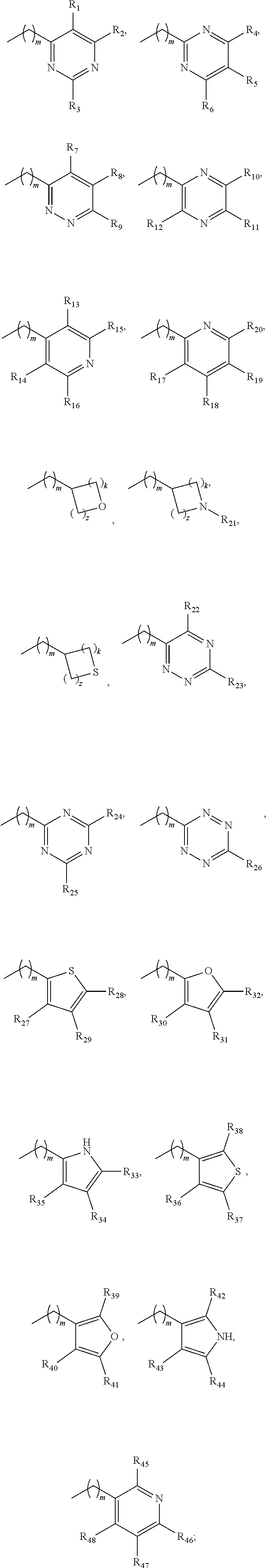

L₁, L₂, L₃, L₄, L₅, L₆, R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₁, R₄₂, R₄₃, R₄₄, R₄₅, R₄₆, R₄₇, R₄₈ at each occurrence represent H, F, Cl, Br, —CN, —CH₃, —CF₃, —OCH₃, —OCF₃ or Ph;
m and n represent a number of CH₂, and are an integer from 1 to 10;
k and z represent a number of CH₂, and are an integer from 0 to 6;
A is

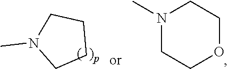

and p represents a number of CH₂, and is 1, 2 or 3; and
X is O or S.

The compound is one of the following compounds:

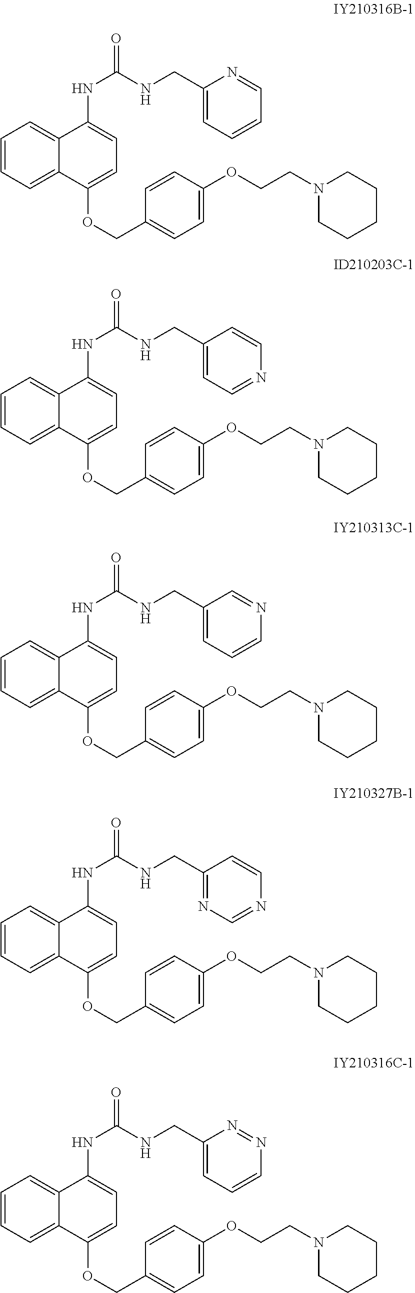 [SMILES: O=C(NCc1ccccn1)Nc1ccc(OCc2ccc(OCCN3CCCCC3)cc2)c2ccccc12 ; O=C(NCc1ccncc1)Nc1ccc(OCc2ccc(OCCN3CCCCC3)cc2)c2ccccc12 ; O=C(NCc1cccnc1)Nc1ccc(OCc2ccc(OCCN3CCCCC3)cc2)c2ccccc12 ; O=C(NCc1ccncn1)Nc1ccc(OCc2ccc(OCCN3CCCCC3)cc2)c2ccccc12 ; O=C(NCc1cccnn1)Nc1ccc(OCc2ccc(OCCN3CCCCC3)cc2)c2ccccc12]

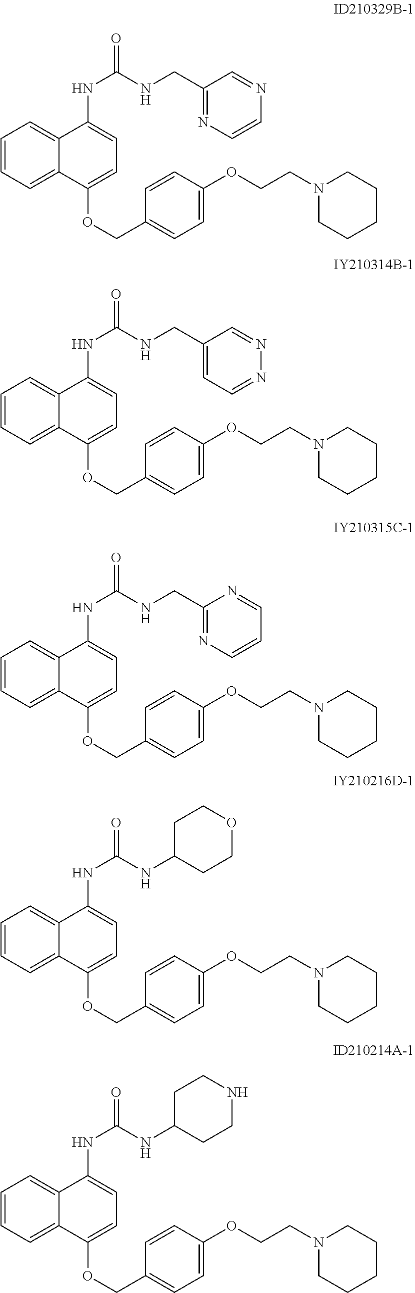 [SMILES: O=C(NCc1cnccn1)Nc1ccc(OCc2ccc(OCCN3CCCCC3)cc2)c2ccccc12 ; O=C(NCc1ccnnc1)Nc1ccc(OCc2ccc(OCCN3CCCCC3)cc2)c2ccccc12 ; O=C(NCc1ncccn1)Nc1ccc(OCc2ccc(OCCN3CCCCC3)cc2)c2ccccc12 ; O=C(Nc1ccc(OCc2ccc(OCCN3CCCCC3)cc2)c2ccccc12)NC1CCOCC1 ; O=C(Nc1ccc(OCc2ccc(OCCN3CCCCC3)cc2)c2ccccc12)NC1CCNCC1]

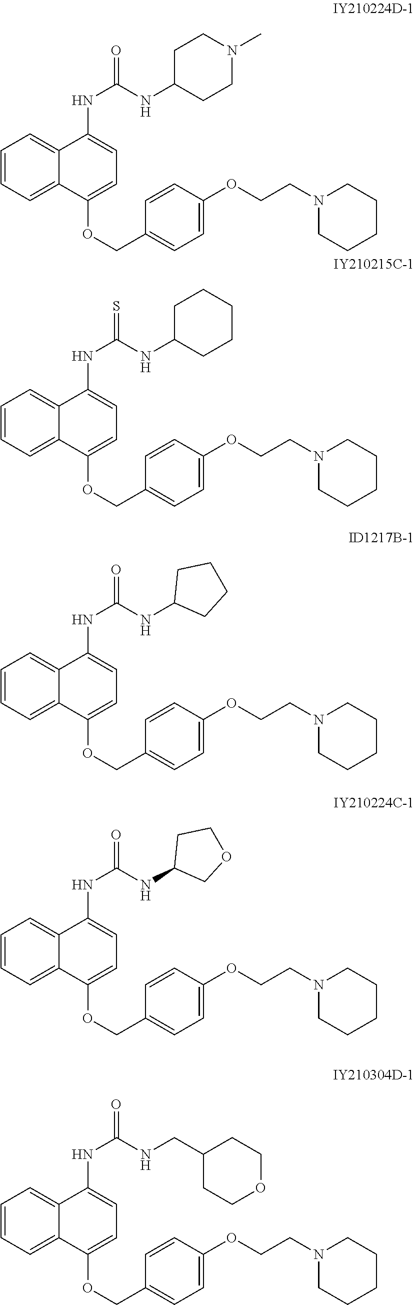 [SMILES: CN1CCC(NC(=O)Nc2ccc(OCc3ccc(OCCN4CCCCC4)cc3)c3ccccc23)CC1 ; S=C(Nc1ccc(OCc2ccc(OCCN3CCCCC3)cc2)c2ccccc12)NC1CCCCC1 ; O=C(Nc1ccc(OCc2ccc(OCCN3CCCCC3)cc2)c2ccccc12)NC1CCCC1 ; O=C(Nc1ccc(OCc2ccc(OCCN3CCCCC3)cc2)c2ccccc12)N[C@H]1CCOC1 ; O=C(NCC1CCOCC1)Nc1ccc(OCc2ccc(OCCN3CCCCC3)cc2)c2ccccc12]

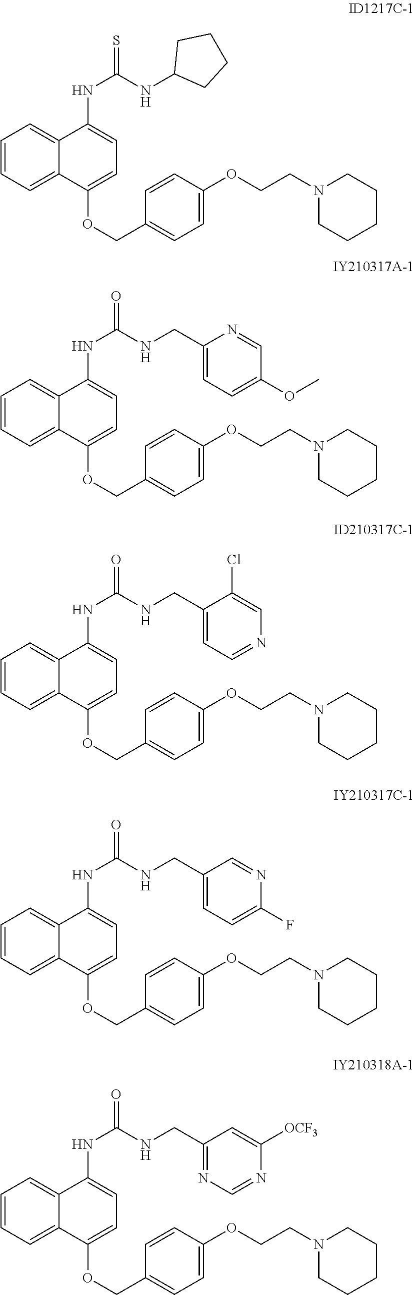 [SMILES: S=C(Nc1ccc(OCc2ccc(OCCN3CCCCC3)cc2)c2ccccc12)NC1CCCC1 ; COc1ccc(CNC(=O)Nc2ccc(OCc3ccc(OCCN4CCCCC4)cc3)c3ccccc23)nc1 ; O=C(NCc1ccncc1Cl)Nc1ccc(OCc2ccc(OCCN3CCCCC3)cc2)c2ccccc12 ; O=C(NCc1ccc(F)nc1)Nc1ccc(OCc2ccc(OCCN3CCCCC3)cc2)c2ccccc12 ; O=C(NCc1cc(OC(F)(F)F)ncn1)Nc1ccc(OCc2ccc(OCCN3CCCCC3)cc2)c2ccccc12]

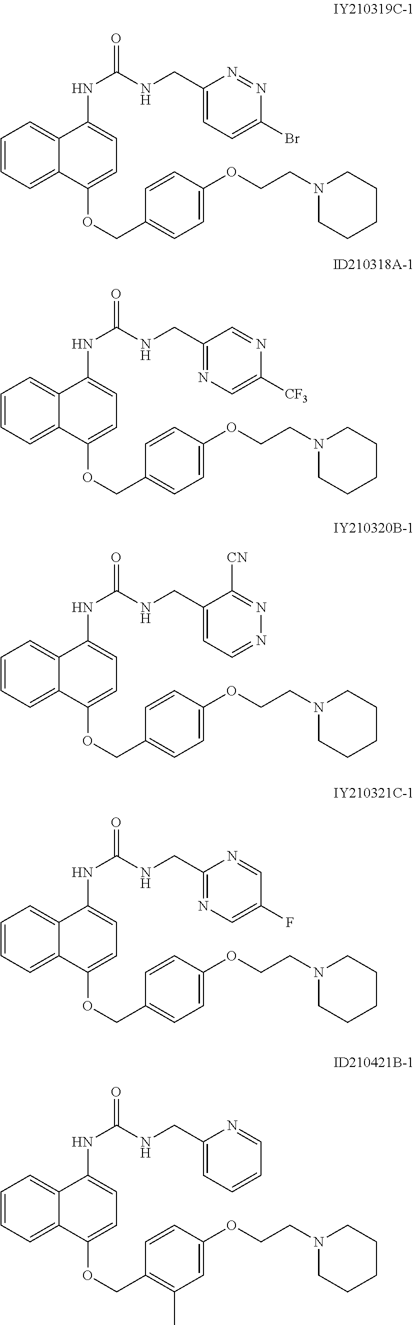 [SMILES: O=C(NCc1ccc(Br)nn1)Nc1ccc(OCc2ccc(OCCN3CCCCC3)cc2)c2ccccc12 ; O=C(NCc1cnc(C(F)(F)F)cn1)Nc1ccc(OCc2ccc(OCCN3CCCCC3)cc2)c2ccccc12 ; N#Cc1nnccc1CNC(=O)Nc1ccc(OCc2ccc(OCCN3CCCCC3)cc2)c2ccccc12 ; O=C(NCc1ncc(F)cn1)Nc1ccc(OCc2ccc(OCCN3CCCCC3)cc2)c2ccccc12 ; Cc1cc(OCCN2CCCCC2)ccc1COc1ccc(NC(=O)NCc2ccccn2)c2ccccc12]

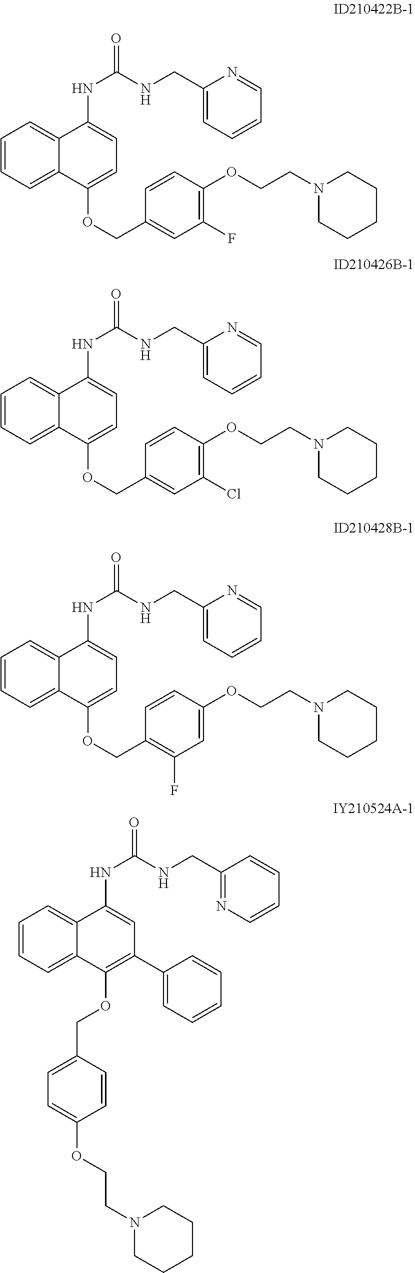 [SMILES: O=C(NCc1ccccn1)Nc1ccc(OCc2ccc(OCCN3CCCCC3)c(F)c2)c2ccccc12 ; O=C(NCc1ccccn1)Nc1ccc(OCc2ccc(OCCN3CCCCC3)c(Cl)c2)c2ccccc12 ; O=C(NCc1ccccn1)Nc1ccc(OCc2ccc(OCCN3CCCCC3)cc2F)c2ccccc12 ; O=C(NCc1ccccn1)Nc1cc(-c2ccccc2)c(OCc2ccc(OCCN3CCCCC3)cc2)c2ccccc12]

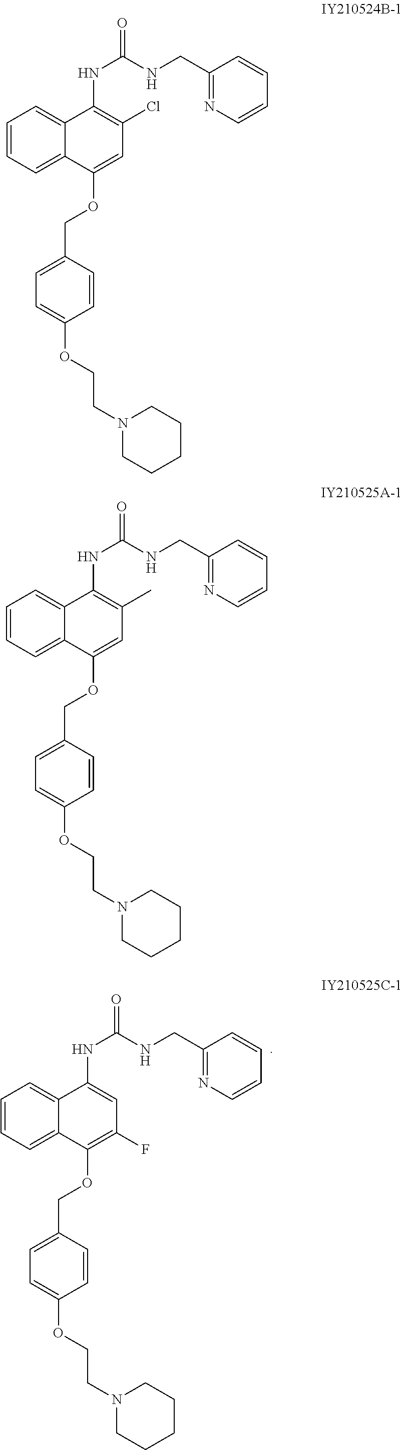

The disclosure also provides a biologically acceptable salt, being formed by contacting the naphthylurea compound with at least an acid selected from the group consisting of acetic acid, dihydrofolic acid, benzoic acid, citric acid, sorbic acid, propionic acid, oxalic acid, fumaric acid, maleic acid, hydrochloric acid, malic acid, phosphoric acid, sulfite, sulfuric acid, vanillic acid, tartaric acid, ascorbic acid, boric acid, lactic acid, and ethylenediaminetetraacetic acid.

A method for preparing the naphthylurea compound comprises:
1) dissolving

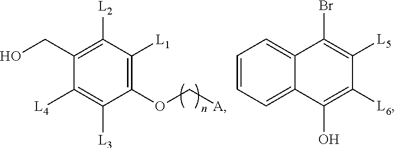

and triphenylphosphine in tetrahydrofuran to yield a mixture, adding diisopropyl azodicarboxylate at −5-5° C. to the mixture, and stirring at room temperature, to yield

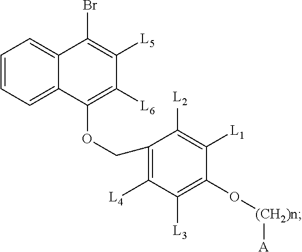

2) dissolving

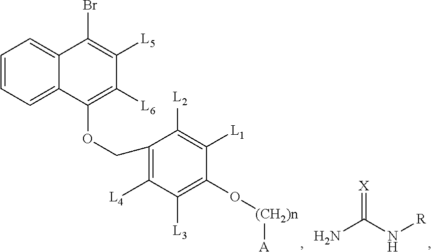

and potassium tert-butoxide in methylbenzene, and adding Pd₂(dba)₃ and 4,5-bis(diphenylphosphino)-9,9-dimethylxanthene at nitrogen atmosphere to the methylbenzene, allowing to react at 110° C., to yield

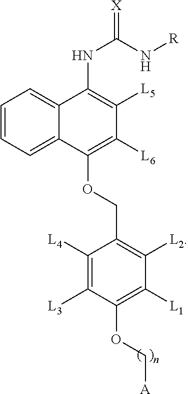

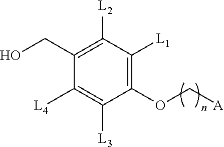

is prepared as follows:
a) dissolving

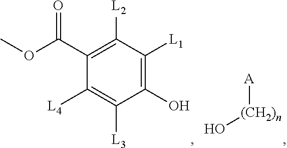

and triphenylphosphine in tetrahydrofuran, and adding diisopropyl azodicarboxylate to a resulting mixture at −5-5° C. under protective atmosphere, and stirring at room temperature, to yield

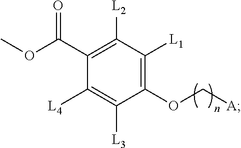

and
b) dissolving

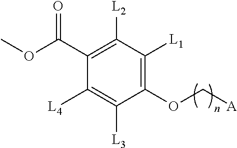

in tetrahydrofuran, adding lithium aluminum hydride in batches at −5-5° C., and stirring at room temperature, to yield

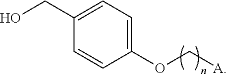

In a class of this embodiment, in 1), a molar ratio of

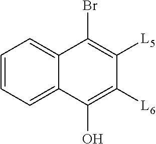

to

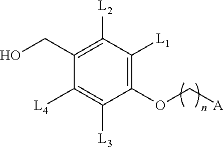

to triphenylphosphine to diisopropyl azodicarboxylate is 1:1:1.2:1.2; in 2), a molar ratio of

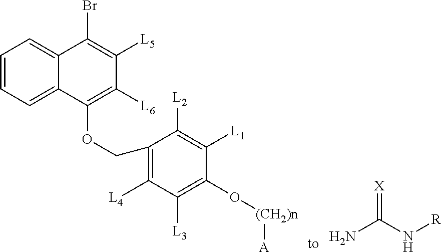

to H to potassium tert-butoxide to 4,5-bis(diphenylphosphino)-9,9-dimethylxanthene is 1:1:1.3:0.05:0.1.\

In a class of this embodiment, in a), a molar ratio of

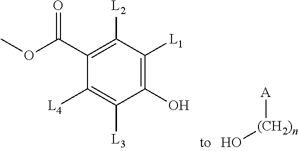

to triphenylphosphine to diisopropyl azodicarboxylate is 1:1.2:1.2:1.2; and in b), a molar ratio of

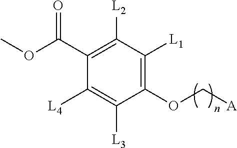

to lithium aluminum hydride is 1:1.

Further provided is a method for treating a tumor comprising administering a patient in need thereof the naphthylurea compound or a biologically acceptable salt thereof, the tumor being a JAKs or STAT3 signaling-related disease.

In a class of this embodiment, the tumor is liver cancer, breast cancer, lung cancer, or leukemia.

Another objective of the disclosure is to provide a small molecule compound with anti-tumor activity.

The tumor is highly proliferative or has a high level of JAK2/STAT3 expression; the tumor includes, but is not limited to, liver cancer, breast cancer, lung cancer, leukemia, and colon cancer.

Specifically, the disclosure synthesizes the novel naphthylurea compounds IY210216D-1, ID210203C-1 and IY210316B-1; MTT assay is used to measure the anticancer activity of the compounds IY210216D-1, ID210203C-1 and IY210316B-1; flow cytometry is used to analyze the cell cycle and apoptosis of the tumor cells treated with the compounds. Immunoblotting and other methods are used to test the inhibitory effect of the compounds on JAK2/STAT3 signal.

The results indicate that the compounds IY210216D-1, ID210203C-1 and IY210316B-1 inhibit proliferation and development of tumor cells within liver cancer and breast cancer, causing the tumor cells to be arrested in the G1/S or G2/M phase of the cell cycle and undergo apoptosis.

### DETAILED DESCRIPTION OF THE DRAWINGS

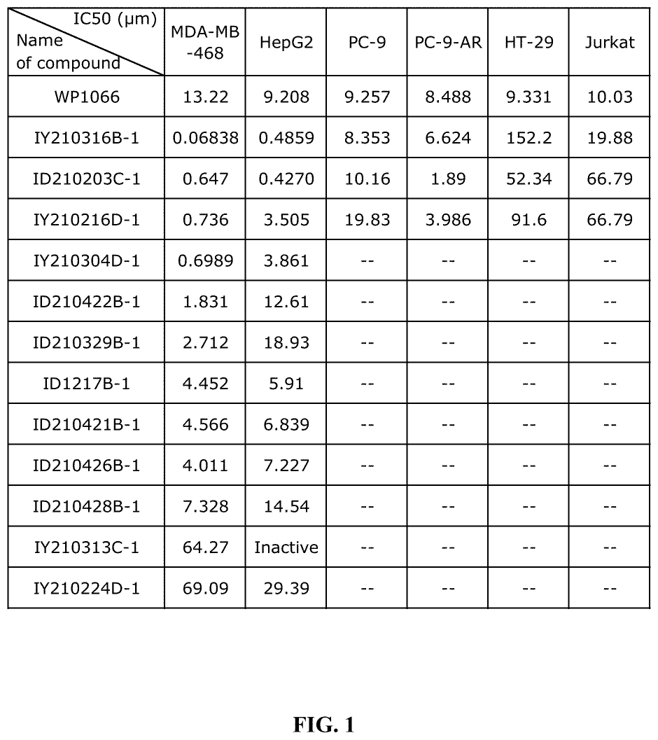

FIG. 1 shows the test results of half inhibition rate (IC50 value) of the compounds IY210216D-1, ID210203C-1 and IY210316B-1 on breast cancer cell MDA-MB-468, liver cancer cell HepG2, lung cancer cell PC9, alfatinib resistant lung cancer cell PC9-AR, colon cancer cell HT29 and leukemia cell Jurkat;

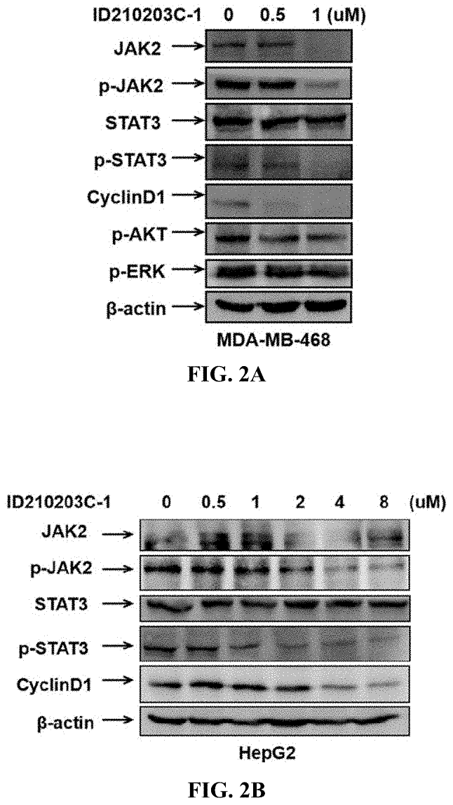

FIG. 2A shows the regulation effect on the expression of JAK2/STAT3 signal axis and other proteins in MDA-MB-468 cells after being treated by 0, 0.5, and 1 μM of the compound ID210203C-1 for 48 hours through Western blot detection;

FIG. 2B shows the regulation effect on the expression of JAK2/STAT3 signal axis and other proteins in HepG2 cells after being treated by 0, 0.5, 1, 2, 4 and 8 μM of the compound ID210203C-1 for 48 hours through Western blot detection;

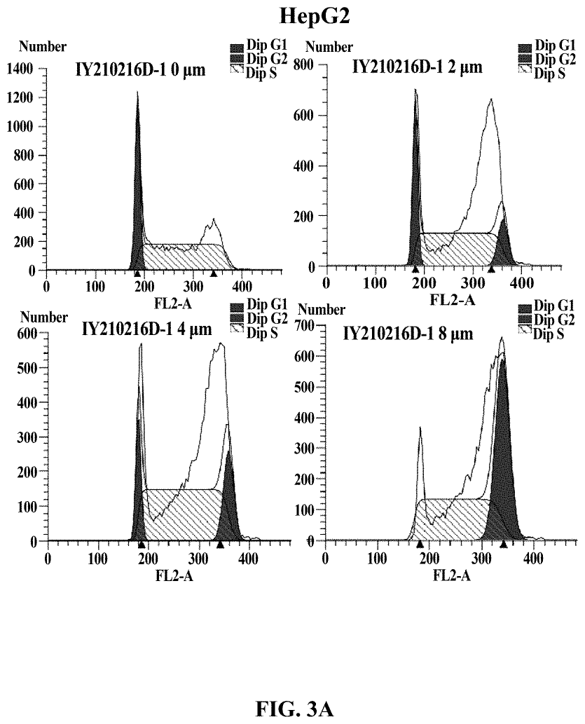

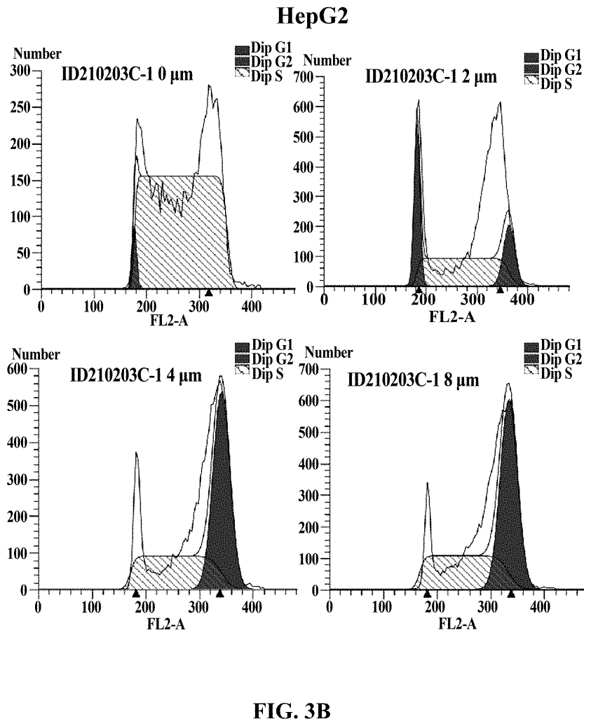

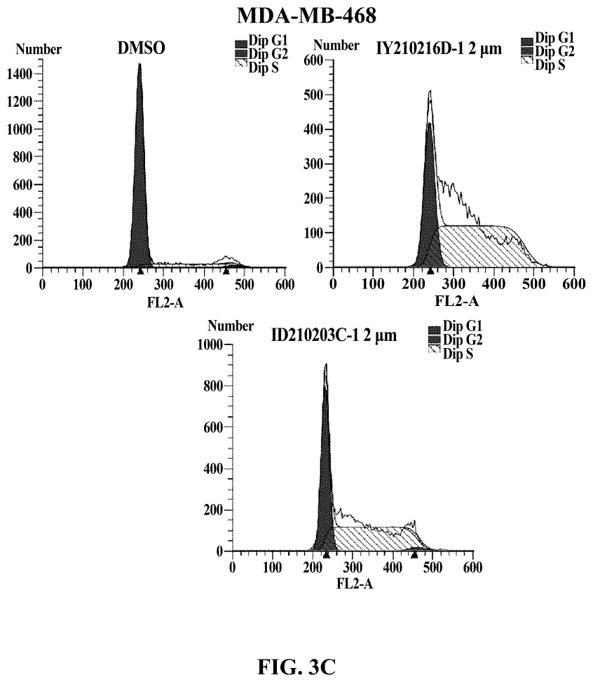

FIG. 3A shows the effect on the cycle progression of HepG2 cells after being treated by 0, 2, 4 and 8 μM of the compound IY210216D-1 for 48 hours through flow cytometry detection;

FIG. 3B shows the effect on the cycle progression of HepG2 cells after being treated by 0, 2, 4 and 8 μM of the compound ID210203C-1 for 48 hours through flow cytometry detection;

FIG. 3C shows the effect on the cycle progression of HepG2 cells after being treated by 2 μM of the compounds IY210216D-1 and ID210203C-1 for 48 hours through flow cytometry detection, with DMSO only as a blank control;

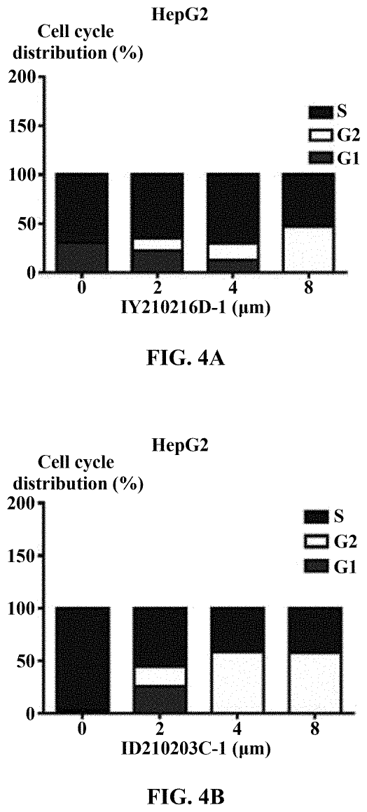

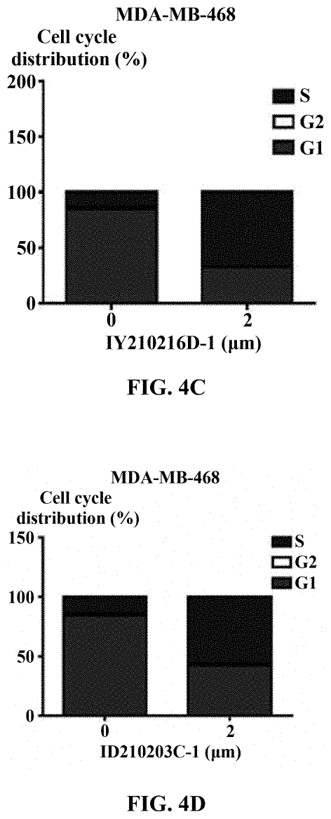

FIG. 4A is a quantitative analysis of the results of FIG. 3A;

FIG. 4B is a quantitative analysis of the results of FIG. 3B;

FIG. 4C is a quantitative analysis of the results of the experimental hole IY210216D-1 and the control hole in FIG. 3C;

FIG. 4D is a quantitative analysis of the results of the ID210203C-1 experimental hole and the control hole in FIG. 3C;

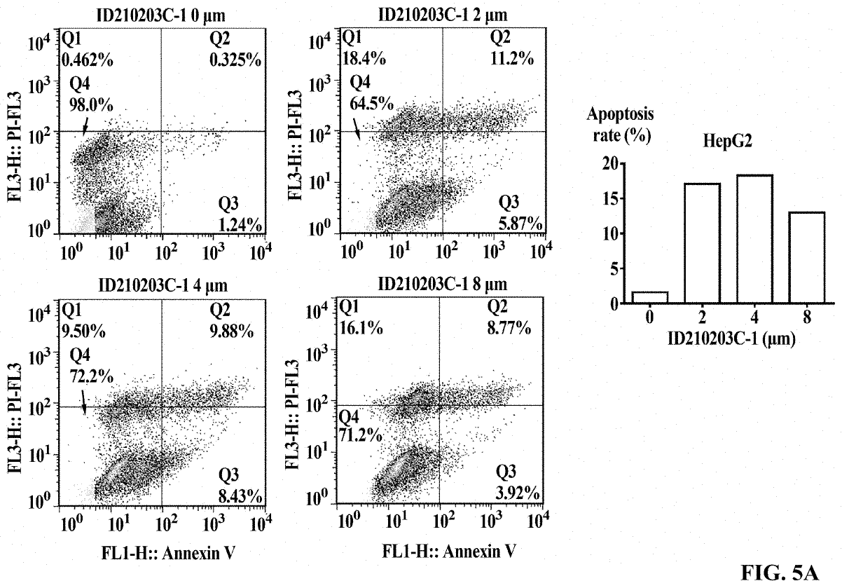

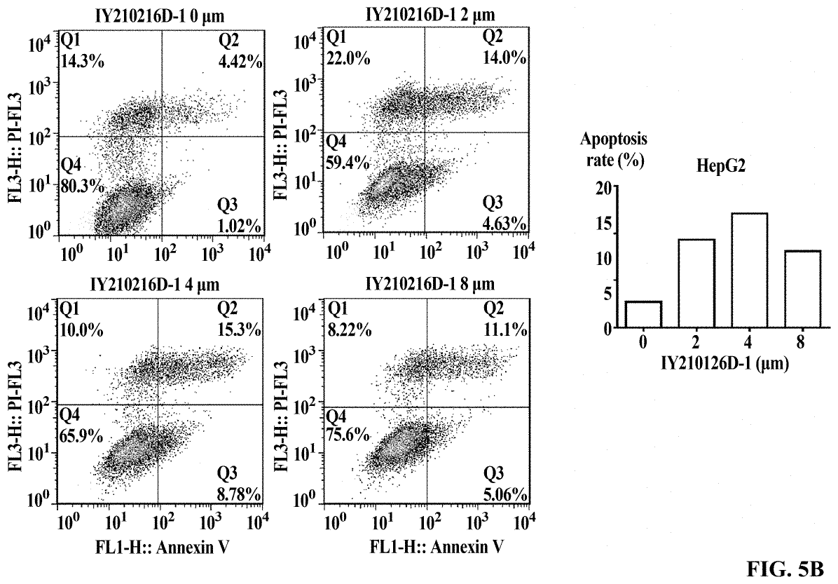

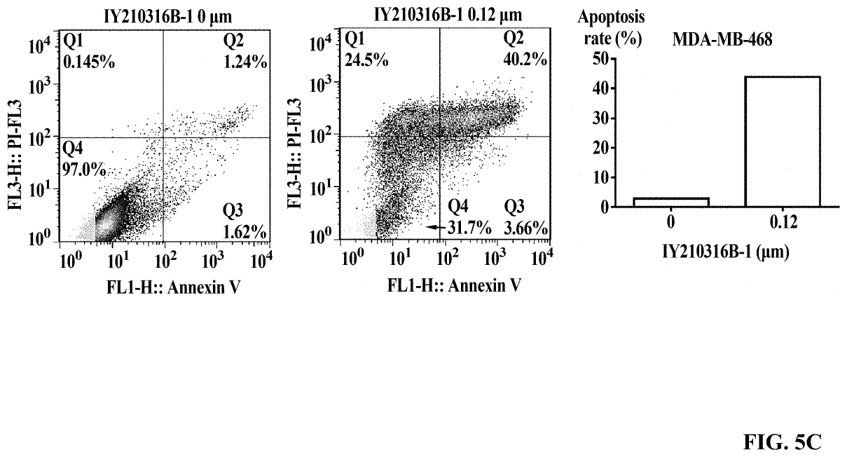

FIG. 5A shows the effect on the apoptosis of HepG2 cells after being treated by 0, 2, 4 and 8 μM of the compound ID210203C-1 for 48 hours through flow cytometry detection;

FIG. 5B shows the effect on the apoptosis of HepG2 cells after being treated by 0, 2, 4 and 8 μM of the compound IY210216D-1 for 48 hours through flow cytometry detection;

FIG. 5C shows the effect on the apoptosis and quantitative analysis of HepG2 cells after being treated by 0 and 0.12 μM of the compound IY210316B-1 for 48 hours through flow cytometry detection; and

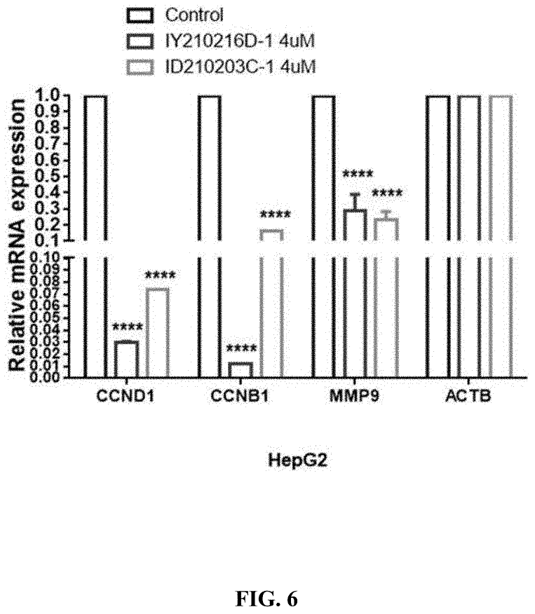

FIG. 6 shows the effect of the compounds IY210216D-1 and ID210203C-1 on the mRNA level of cell cycle and metastasis related molecules detected by Q-PCR.

### DETAILED DESCRIPTION

To further illustrate the disclosure, embodiments detailing a naphthylurea compound are described below. It should be noted that the following embodiments are intended to describe and not to limit the disclosure.

In a method for synthesizing the naphthylurea compound having the formula I, all raw materials are commercially available or prepared by those skilled in the prior arts. In the disclosure, the intermediates, raw materials, reagents, and reaction conditions are changed by the person skilled in the art.

In the disclosure, (i) the temperature is seen in units of degree Celsius or ° C.; and the synthesis method is performed at room temperature ranging from 20° C. to 30° C.; (ii) a common method is used to dry the organic solvent; a rotary evaporator is used to remove solvent from a sample through evaporation under reduced pressure; the maximum temperature for a bath is 50° C.; a developing solvent and an eluting solvent are added in a volume ratio; (iii) thin layer chromatography (TLC) is used to monitor the progress of chemical reaction; (iv) a final product is obtained and produces enough signals in a 1H NMR spectrum.

### Example 1 Compound Synthesis

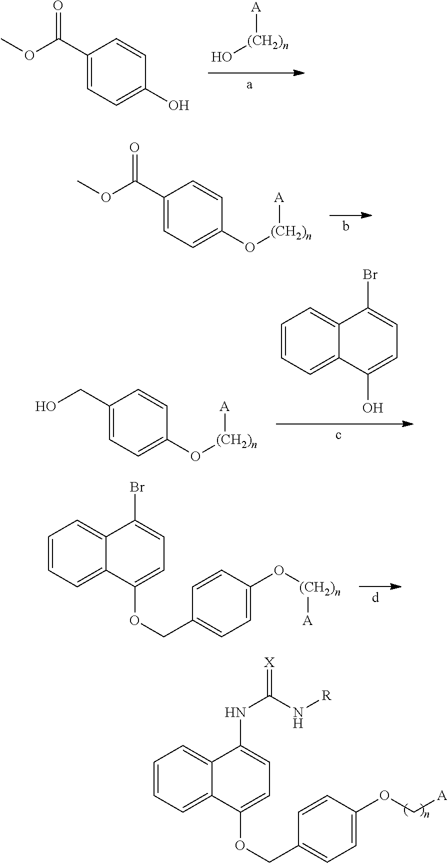

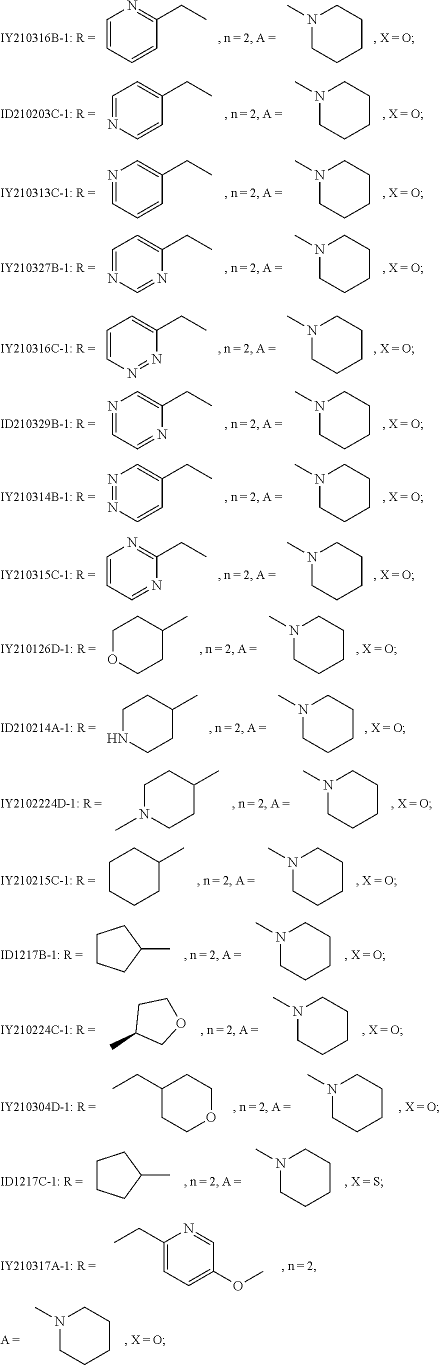

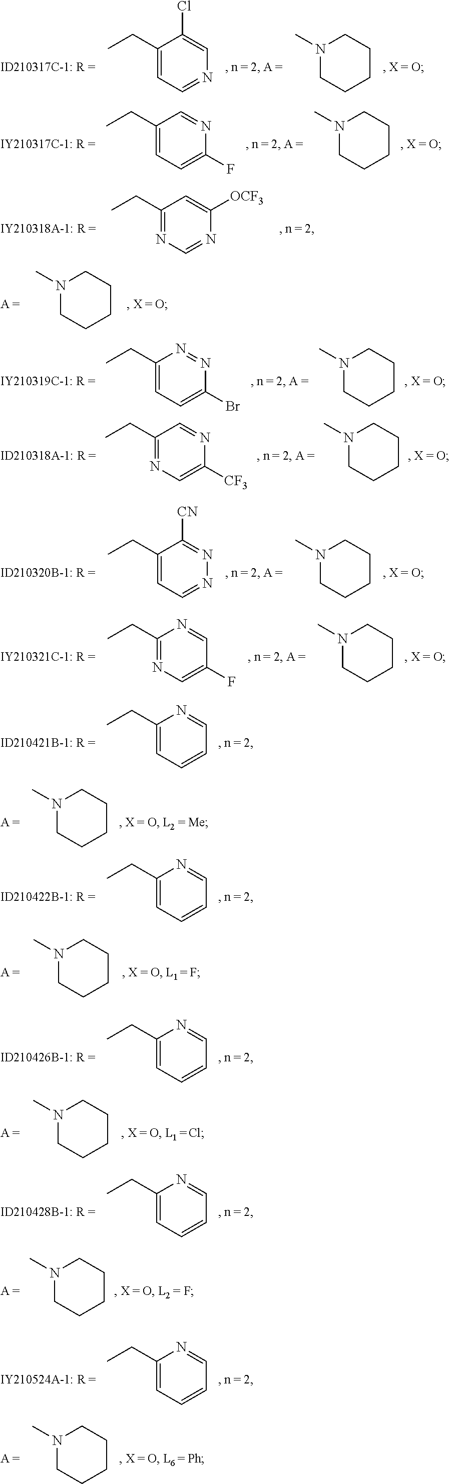

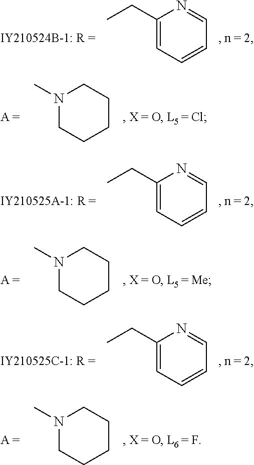

For example, the naphthylurea compound IY210316B-1 has the following formula:

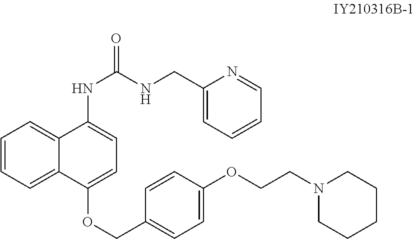 [SMILES: O=C(NCc1ccccn1)Nc1ccc(OCc2ccc(OCCN3CCCCC3)cc2)c2ccccc12]

The naphthylurea compound IY210316B-1 is named 1-(4-((4-(2-(piperidin-1-yl)ethoxy)benzyl)oxy)naphthalen-1-yl)-3-(pyridin-2-ylmethyl)urea.

The naphthylurea compound IY210316B-1 is synthesized by the following route:

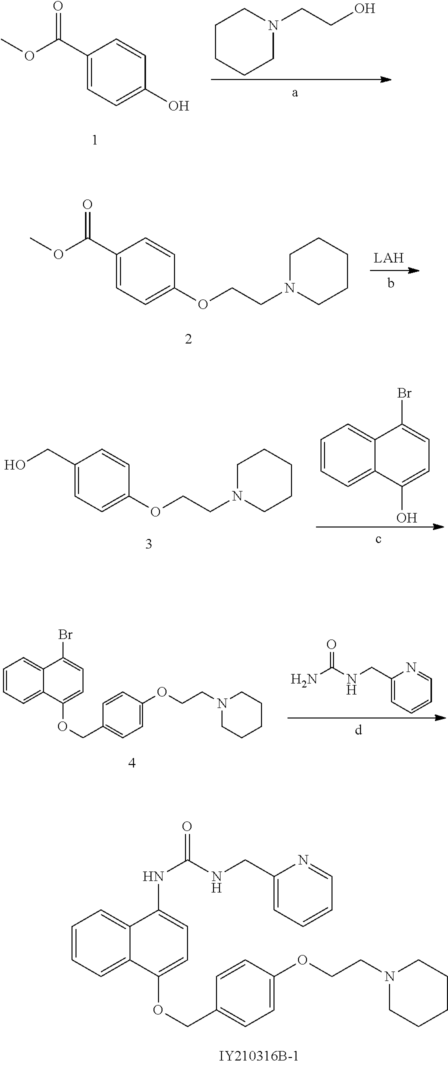

### Step 1. Preparation of methyl 4-(2-(piperidin-1-yl)ethoxy)benzoate (Compound 2)

1.0 g of methyl 4-hydroxybenzoate (Compound 1, 6.57 mmol, 1.0 eq), 1.02 g of N-hydroxyethylpiperidine (7.89 mmol, 1.2 eq) and 2.07 g of triphenylphosphine (7.89 mmol, 1.2 eq) was dissolved in 30 mL of anhydrous tetrahydrofuran (THF) to yield a mixture; the mixture was cooled to 0° C.; 1.59 g of diisopropyl azodicarboxylate (7.89 mmol, 1.2 eq) was added dropwise to the cooled mixture under nitrogen and allowed to react at room temperature for 16 h; when a thin layer chromatography (TLC) plate showed that no more starting materials are left in the reaction time, the resulting mixture was concentrated under reduced pressure to remove THF, and a solid is formed; the solid was dissolved in ethyl acetate to form a solution; the pH of the solution was adjusted to 1 with 1N hydrochloric acid; the solution was extracted three times with ethyl acetate; the pH of the aqueous phase was adjusted to 8 with sodium bicarbonate; the aqueous phase was extracted three times with ethyl acetate; the organic phase was dried and spin-dried to yield 1.5 g of a white solid; the white solid is methyl 4-(2-(piperidin-1-yl)ethoxy)benzoate (Compound 2) in 86.7% yield).

1H NMR (CDCl3, 300 MHz) δ: 8.0 (d, J=9.0 Hz, 2H), 6.93 (d, J=9.0 Hz, 2H), 4.17 (t, J=6.0 Hz, 2H), 3.90 (s, 3H), 2.82 (t, J=6.0 Hz, 2H), 2.58-2.55 (m, 4H), 1.66-1.61 (m, 4H), 1.50 (t, J=3.0 Hz, 2H)

### Step 2. Preparation of (4-(2-(piperidin-1-yl)ethoxy)phenyl)methanol (Compound 3)

1.00 g of methyl 4-(2-(piperidin-1-yl)ethoxy)benzoate (Compound 2, 3.80 mmol, 1.0 eq) was dissolved in 40 mL of anhydrous THF to yield a solution; the solution was cooled to 0° C.; 144 mg of lithium aluminum hydride (3.80 mmol, 1.0 eq) was added in batches to the cooled solution to form a mixture; the mixture temperature was naturally raised to room temperature and the mixture was allowed to react at room temperature for 0.5 h; the TLC plate showed that no more starting materials were left in the reaction mixture and new spots were visualized; the reaction mixture was cooled to 0° C.; 1 mL of NaOH (15 wt %) aqueous solution and 1 mL of water were added successively; the resulting mixture was filtered with diatomaceous earth; the filtrate was spin-dried to yield 680 mg of a white solid; the white solid is (4-(2-(piperidin-1-yl)ethoxy)phenyl)methanol (Compound 3) in 88.7% yield.

¹H NMR (CDCl₃, 300 MHz) δ: 7.30 (d, J=6.0 Hz, 2H), 6.92 (d, J=6.0 Hz, 2H), 4.64 (s, 2H), 4.17 (t, J=6.0 Hz, 2H), 2.98 (t, J=6.0 Hz, 2H), 2.74 (m, 4H), 1.89-1.86 (m, 6H)

### Step 3. Preparation of 1-(2-(4-(((4-bromonaphthalen-1-yl)oxy)methyl)phenoxy)ethyl)piperidine (Compound 4)

1.05 g of (4-(2-(piperidin-1-yl)ethoxy)phenyl)methanol (Compound 3) (4.48 mmol, 1.0 eq), 4-bromo-1-naphthol (1.0 g, 4.48 mmol, 1.0 eq), and triphenylphosphine (1.41 g, 5.38 mmol, 1.2 eq) were dissolved in 50 mL of anhydrous THE to form a solution; the solution was cooled to 0° C.; and then diisopropyl azodicarboxylate (1.09 g, 5.38 mmol, 1.2 eq) was added slowly to the solution, and allowed to react at room temperature for 12 hrs. When the TLC plate showed that no more starting materials were left in the reaction time, 100 mL of saturated ammonium chloride aqueous solution was added to form a resulting mixture; the resulting mixture was extracted three times with ethyl acetate (each time 100 mL); the organic phases were mixed together; the mixed organic phase was dried with anhydrous sodium sulfate, spin-dried, and passes through the spin column (a ratio of the volume of dichloromethane to methanol is (60:1)-(20:1)) to yield 710 mg of a yellow solid; the yellow solid is 1-(2-(4-(((4-bromonaphthalen-1-yl)oxy)methyl)phenoxy)ethyl)piperidine (Compound 4) in 47.6% yield.

### Step 4 1-benzyl-3-(4-((4-(2-(piperidin-1-yl)ethoxy)benzyl)oxy)naphthalen-1-yl)urea (IY210316B-1)

1-(2-(4-(((4-bromonaphthalen-1-yl)oxy)methyl)phenoxy)ethyl)piperidine (Compound 4, 200 mg, 0.45 mmol, 1.0 eq), 1-(pyridin-2-ylmethyl) urea (68.6 mg, 0.45 mmol, 1.0 eq) and potassium tert-butanol (66.3 mg, 0.59 mmol, 1.3 eq) were dissolved in 50 mL of toluene, and Pd₂(dba)₃ (50 mg, 0.03 mmol, 0.05 eq), Xanthos (4,5-bis (diphenylphosphine)-9,9-dimethyloxacene, 15 mg, 0.06 mmol, 0.1 eq) were added successively under nitrogen protection to the resulting mixture and allowed to react for 12 hours at 110° C. When the TLC plate showed that no more starting materials were left in the reaction time, the product was spin-dried, and passes the elute through the spin column (a ratio of the volume of dichloromethane to methanol is (50:1)-(15:1)) to yield 210 mg of a brown solid; the brown solid is 1-benzyl-3-(4-((4-(2-(piperidin-1-yl)ethoxy)benzyl)oxy)naphthalen-1-yl)urea (IY210316B-1) in 77.8% yield.

¹H NMR (DMSO-d6, 400 MHz) δ: 8.32 (s, 1H), 8.19 (d, J=8.0 Hz, 1H), 8.01 (d, J=8.0 Hz, 2H), 7.68 (d, J=8.0 Hz, 2H), 7.58-7.26 (m, 8H), 7.05-6.98 (m, 3H), 6.82 (m, 1H), 5.20 (s, 2H), 4.34 (d, J=4.0 Hz, 2H), 4.11 (m, 2H), 2.52 (m, 2H), 1.53 (m, 4H), 1.40 (m, 2H), 1.39-1.20 (m, 2H).

The other compounds are synthesized according to the above method in Example 1, except for the following differences:

in step 4, the urea is substituted by corresponding R-group, or in step 1, 4-hydroxybenzoate methyl ester is replaced by L₁, L₂, L₃ or L₄ substituted 4-hydroxybenzoate methyl ester, or in step 3, 4-bromo-1-naphthol is replaced by L₅ or L₆ substituted 4-bromo-1-naphthol.

### Example 2

Inhibitory Effects of the Compounds IY210216D-1, ID210203C-1 and IY210316B-1 on Proliferation of Breast Cancer and Liver Cancer

Tumor cells in logarithmic growth phase were collected; the cell suspension was diluted to a density of 5×10⁴ cells/mL; 100 uL of the cell suspension was transferred to each hole of a 96-well plate; DMSO was used as a solvent for negative control; (2E)-3-(6-bromo-2-pyridyl)-2-cyano-N-[(1S)-1-phenylethyl]-2-acrylamide (WP1066CAS: 857064-38-1, with a formula

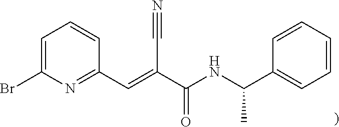 [SMILES: C[C@H](NC(=O)/C(C#N)=C/c1cccc(Br)n1)c1ccccc1]

was used as a positive control; the naphthylurea compounds IY210216D-1, ID210203C-1 and IY210316B-1 were diluted with DMSO and added into the 96-well plate to a final concentration of 0.1, 0.3, 1, 3, 10, 30, 100 and 300 μmol/L; the 96-well plate was incubated for 48 h; 10 μL of MTT solvent (5 mg/mL) was added into each well; the 96-well plate was incubated at 37° C. for 4 h; a culture supernatant was discarded; 150 μL of DMSO was added into each well; the 96-well plate was shaken for 10 min on a plate shaker; an optical density (OD) of the resulting product was measured at a wavelength of 490 nm by an ELISA reader. Test results were recorded. A cell growth curve was drawn with the dosage of each compound as abscissa and the absorbance value as ordinate. The statistical results of the half inhibition rate (IC50 value) of tumor cells by the compounds IY210216D-1, ID210203C-1 and IY210316B-1 were shown in FIG. 1.

As shown in FIG. 1, compared with the positive control drug WP1066, the compounds IY210216D-1, ID210203C-1 and IY210316B-1 have good inhibitory effects on the proliferation of tumor cells such as breast cancer and liver cancer, especially in breast cancer and liver cancer cells. The anti-tumor effects of the three compounds in breast cancer and liver cancer are particularly emphasized.

### Example 3

Regulatory Effect of ID210203C-1 on the Protein Expression of JAK2/STAT3 Signal Axis Through Western Blot Detection

MDA-Mb-468 or HepG2 cells in logarithmic growth phase were inoculated in a 6-well plate, with 8×10⁵ cells per well. After cell adhesion, the compound ID210203C-1 was added until a final concentration thereof was 0, 0.5, 1 or 0, 0.5, 1, 2, 4 and 8 μM respectively. After about 48 hours, the cells were lysed with RIPA lysate and proteins were collected for Western blot analysis. The protein expressions were respectively detected through the anti-JAK2, p-JAK2, STAT3, p-STAT3, CyclinD1, p-AKT, p-ERK and β-actin antibodies.

As shown in FIGS. 2A-2B, compared with the solvent control, the treatment with ID210203C-1 can significantly inhibit the expression level of p-JAK2, p-STAT3 and CyclinD1 in a dose-dependent manner. It shows that the compound ID210203C-1 can targeted inhibit the phosphorylation of JAK2 and STAT3 proteins and the expression of downstream target genes.

### Example 4

The Compounds IY210216D-1 and ID210203C-1 Significantly Induce the Cell Cycle Arrest in Breast Cancer and Liver Cancer Cells

MDA-MB-468 or HepG2 cells were harvested during log phase, digested, centrifuged and prepared into a single cell suspension; the number of the cells in the single cell suspension was counted; the cells were seeded into a 12-well plate, with 2×10⁵ cells per well; three wells were used as a parallel control design; 16 hours after seeding, the cells were treated with the compounds. With DMSO as a solvent, the final concentrations of the compounds IY210216D-1 and ID210203C-1 in HepG2 cell suspension were 0, 2, 4 and 8 μM, respectively, and the final concentrations of the compounds IY210216D-1 and ID210203C-1 in MDA-MB-468 cell suspension were 0 and 2 μM, respectively. 48 hours later, the cells were digested with trypsin and resuspended; the number of the cells in the cell suspension was counted and diluted to 5×10⁵ cells/mL; after the digestion was completed, the cell suspension was centrifuged; the supernatant was discarded; the pellet was washed twice with PBS (each time the mixture was centrifuged 2000 rpm for 5 min); the supernatant was discarded; a fixative comprising 980 μL of 70% cold ethanol and 20 μL of 5% BSA (a small amount of BSA reduces cellular stress and damage) was added to each microcentrifuge tube, so that the cells were fixed overnight at 4° C.; the fixative is discarded; the cells were washed three times in PBS to remove residual fixative (each time the mixture was centrifuged at 1000 rpm for 3 min); a DNA quantification kit is used to measure the content of DNA according to the following instruction (Suo Laibao, Beijing): each sample was incubated in 100 μL of RNase A at 37° C. for 30 min; 500 μL of PI (propidium iodide) was added to each sample; each sample was incubated at room temperature for 30 min in the dark; the cell cycle was analyzed by a flow cytometry and a ModFit software; and Graphpad prism 6.0 was used to estimate the percentage of a cell population in the different phases of the cell cycle.

FIGS. 3A-3C show the result of the cycle distribution of the HepG2 and MDA-MB-468 cells affected by the compounds IY210216D-1 and ID210203C-1 using ModFit software.

FIGS. 4A-4D are quantitative analysis of the results of FIGS. 3A-3C through Graphpad Prism 6.0. The results in FIGS. 3A-3C and FIGS. 4A-4D show that compared with the solvent control group (DMSO), the compounds IY210216D-1 and ID210203C-1 can induce a significant increase in the G2 phase ratio and a significant decrease in the G1 phase ratio of hepatoma cells. Both the compounds IY210216D-1 and ID210203C-1 can induce a significant increase in the S phase ratio of breast cancer cells and a decrease in the G1 phase ratio.

### Example 5

Induction of Apoptosis in Cancer Cells by Compounds IY210216D-1, ID210203C-1 and IY210316B-1

The MDA-MB-468 or HepG2 cells were harvested during log phase, digested, centrifuged and prepared into a single cell suspension; the number of the cells in the cell suspension was counted; the cells were seeded into a 12-well plate, with 2×10⁵ cells per well; three wells were used as a parallel control design; 16 hours after seeding, the cells were treated with the compounds.

With DMSO as a solvent, the final concentrations of the compounds IY210216D-1 and ID210203C-1 in HepG2 cell suspension were 0, 2, 4 and 8 μM, respectively, and the final concentrations of the compound IY210316B-1 in MDA-MB-468 cell suspension were 0 and 0.12 μM, respectively. 48 hours later, the cells were digested with EDTA-free trypsin and resuspended; the number of the cells in the cell suspension was counted and diluted to 1×10⁶ cells/mL; an annexin V apoptosis detection kit was used according to the following instruction (Suo Laibao, Beijing): the cells were washed twice with 1×PBS (each time the mixture was centrifuged at 6000 rpm for 0.5 min), washed once with 1×Binding buffer (and the mixture was centrifuged at 6000 rpm for 0.5 min); the supernatant was discarded; the cells were resuspended with 500 μL of 1×Binding buffer; 5 μL of Annexin V-FITC was added into each tube, and incubated in the dark for 10 min; 5 μL of PI was added into each tube and incubated in the dark for 5 min; and each tube was then inspected on a machine in the dark.

FIGS. 5A-5C show the effect of the compounds IY210216D-1, ID210203C-1 and IY210316B-1 on tumor cell apoptosis by flow cytometry. The results showed that compared with the control group, the compounds IY210216D-1, ID210203C-1 and IY210316B-1 could induce increased apoptosis in a dose-dependent manner. Especially, the treatment with 0.12 μM of the compound IY210316B-1 for 48 hours can induce 43.86% apoptosis of breast cancer cells.

### Example 6

The Compounds IY210216D-1 and ID210203C-1 Affect the Expression of Cell Cycle Regulatory Molecules and Metastasis Related Genes

HepG2 liver cancer cells were seeded in a 6-well plate, with 1×10⁶ cells per well, and treated with the compounds IY210216D-1 and ID210203C-1 (in 0 and 4 μM concentrations) for 24 h; total RNA was extracted from the HepG2 liver cancer cells by a single-step TRIzol method; the concentration and purity of the total RNA was measured; the total RNA was used as a template; and complementary DNA (cDNA) was synthesized from the RNA template according to the instruction of a reverse transcription kit (Promega); sqRT-PCR and qPCR were used to quantify the expression of the genes CCNB1, CDK1 and SQSTM; and the gene ACTB was used as an internal reference gene for gene expression normalization. Sequences of primers used to quantify gene expression are listed in Table 1.

TABLE 1
Sequences of primers used to quantify gene
expression

| Gene | Sequence |
|---|---|
| CCND1-F | GCTGCGAAGTGGAAACCATC (SEQ ID NO: 1) |
| CCND1-R | CCTCCTTCTGCACACATTTGAA (SEQ ID NO: 2) |
| CDNB1-F | AATAAGGCGAAGATCAACATGGC (SEQ ID NO: 3) |
| CDNB1-R | TTTGTTACCAATGTCCCCAAGAG (SEQ ID NO: 4) |
| MMP9-F | AGACCTGGGCAGATTCCAAAC (SEQ ID NO: 5) |
| MMP9-R | CGGCAAGTCTTCCGAGTAGT (SEQ ID NO: 6) |
| ATCB-F | CATGTACGTTGCTATCCAGGC (SEQ ID NO: 7) |
| ATCB-R | CTCCTTAATGTCACGCACGAT (SEQ ID NO: 8) |

A20 μL reaction mix for qPCR contained:
2 μL of cDNA;
10 μL of 2×SYBR Green Supermix;
1 μL of upstream and downstream primers;
0.3 μL of reference dye;
6.7 μL of water.
Each sample has three technical replicates.
Cycling conditions comprised:
pre-denaturation at 95° C. for 5 min;
denaturation at 95° C. for 15 sec;
annealing at 60° C. for 15 sec; and
extension at 72° C. for 30 sec.

After 40 cycles, the cycle threaded (CT) value of the 3-actin gene was used as an initial value in comparison with the amount of the amplified product.

FIG. 6 shows the effect of the compounds IY210216D-1 and ID210203C-1 on the mRNA level of cell cycle and metastasis related molecules detected by Q-PCR. The results show that after treatment with 0 and 4 μM IY210216D-1 and ID210203C-1 for 24 hours, compared with the expression level of actin gene (ATCB), the expressed mRNA levels of two G2 phase regulators Cyclin D1 (gene name: CCND1) and Cyclin B1 (gene name: CCNB1) are down-regulated by almost 10 folds. It is shown that the compounds IY210216D-1 and ID210203C-1 can induce the cell cycle arrest and inhibit the growth and metastasis of the tumor cells by down-regulating the expression of Cyclin D1, Cyclin B1 and MMP9 at mRNA level.

To sum up, the naphthalurea compound represented by IY210216D-1, ID210203C-1 and IY210316B-1 can significantly inhibit the proliferation and metastasis of breast cancer and liver cancer cells, and induce cell cycle arrest and apoptosis of tumor cells, showing a good anti-cancer effect.

The disclosed compounds are suitable for use in treatment of cancers related to abnormal cell proliferation; specifically, the disclosed compounds are altered into pharmaceutically acceptable salts or mixed with drug carriers to form antitumor drugs.

It will be obvious to those skilled in the art that changes and modifications may be made, and therefore, the aim in the appended claims is to cover all such changes and modifications.

What is claimed is:

1. A naphthylurea compound, having the following formula: 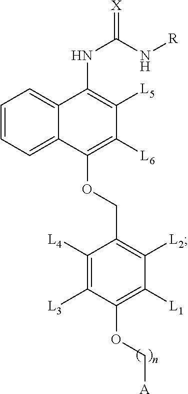
wherein,
R represents 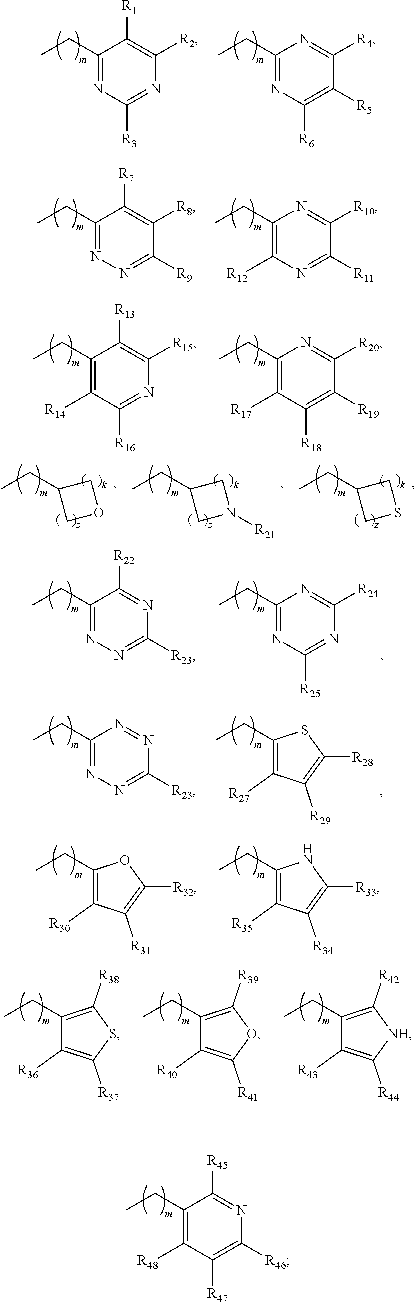
L₁, L₂, L₃, L₄, L₅, L₆, R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₁, R₄₂, R₄₃, R₄₄, R₄₅, R₄₆, R₄₇, R₄₈ at each occurrence represent H, F, Cl, Br, —CN, —CH₃, —CF₃, —OCH₃, —OCF₃ or Ph;
m and n represent a number of CH₂, and are an integer from 1 to 10;
k and z represent a number of CH₂, and are an integer from 0 to 6;
A is 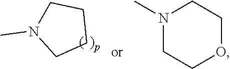
and p represents a number of CH₂, and is 1, 2 or 3; and
X is O or S.

2. The compound of claim 1, being one of the following compounds:

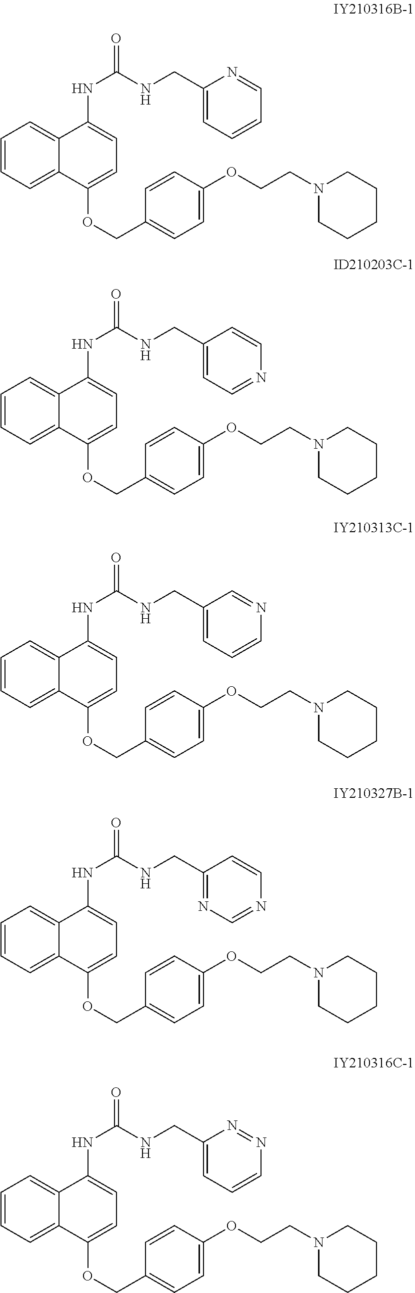 [SMILES: O=C(NCc1ccccn1)Nc1ccc(OCc2ccc(OCCN3CCCCC3)cc2)c2ccccc12 ; O=C(NCc1ccncc1)Nc1ccc(OCc2ccc(OCCN3CCCCC3)cc2)c2ccccc12 ; O=C(NCc1cccnc1)Nc1ccc(OCc2ccc(OCCN3CCCCC3)cc2)c2ccccc12 ; O=C(NCc1ccncn1)Nc1ccc(OCc2ccc(OCCN3CCCCC3)cc2)c2ccccc12 ; O=C(NCc1cccnn1)Nc1ccc(OCc2ccc(OCCN3CCCCC3)cc2)c2ccccc12]

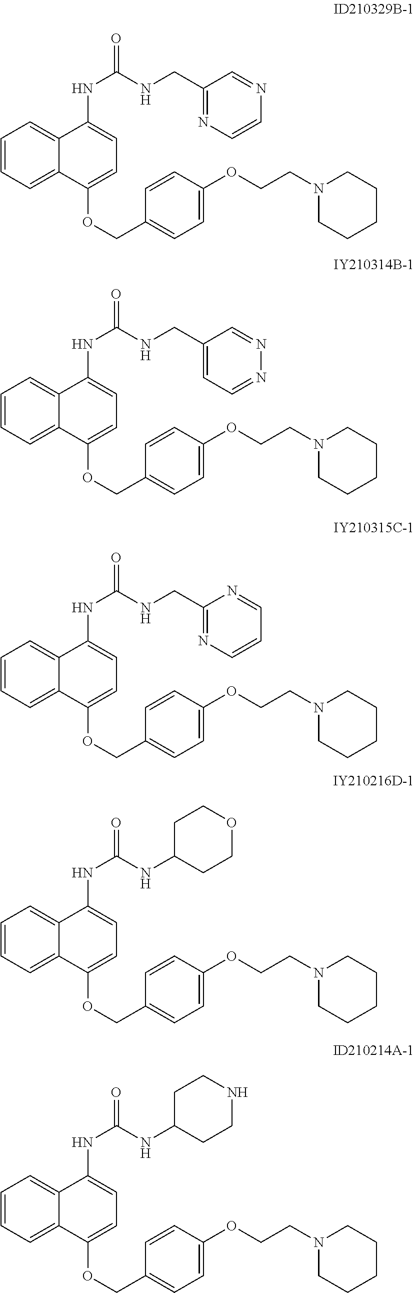 [SMILES: O=C(NCc1cnccn1)Nc1ccc(OCc2ccc(OCCN3CCCCC3)cc2)c2ccccc12 ; O=C(NCc1ccnnc1)Nc1ccc(OCc2ccc(OCCN3CCCCC3)cc2)c2ccccc12 ; O=C(NCc1ncccn1)Nc1ccc(OCc2ccc(OCCN3CCCCC3)cc2)c2ccccc12 ; O=C(Nc1ccc(OCc2ccc(OCCN3CCCCC3)cc2)c2ccccc12)NC1CCOCC1 ; O=C(Nc1ccc(OCc2ccc(OCCN3CCCCC3)cc2)c2ccccc12)NC1CCNCC1]

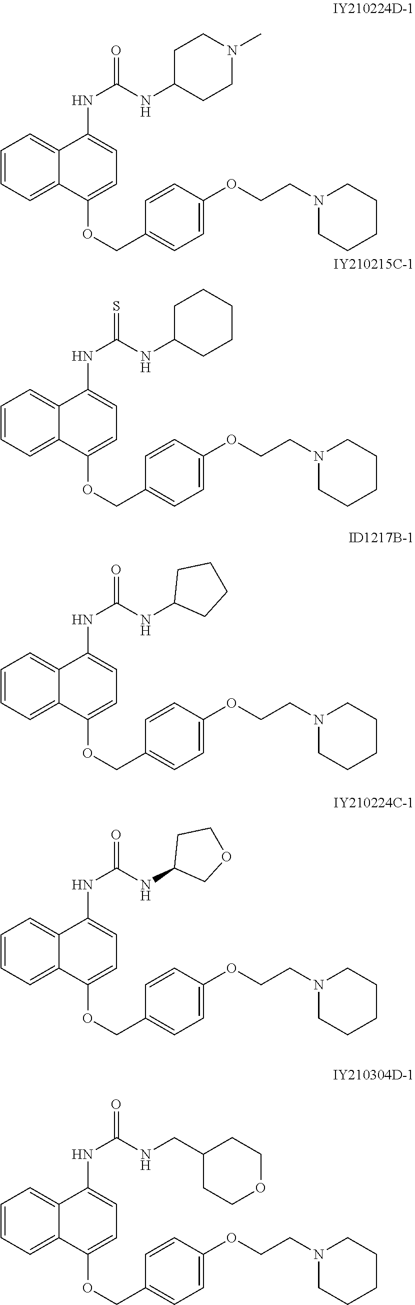 [SMILES: CN1CCC(NC(=O)Nc2ccc(OCc3ccc(OCCN4CCCCC4)cc3)c3ccccc23)CC1 ; S=C(Nc1ccc(OCc2ccc(OCCN3CCCCC3)cc2)c2ccccc12)NC1CCCCC1 ; O=C(Nc1ccc(OCc2ccc(OCCN3CCCCC3)cc2)c2ccccc12)NC1CCCC1 ; O=C(Nc1ccc(OCc2ccc(OCCN3CCCCC3)cc2)c2ccccc12)N[C@H]1CCOC1 ; O=C(NCC1CCOCC1)Nc1ccc(OCc2ccc(OCCN3CCCCC3)cc2)c2ccccc12]

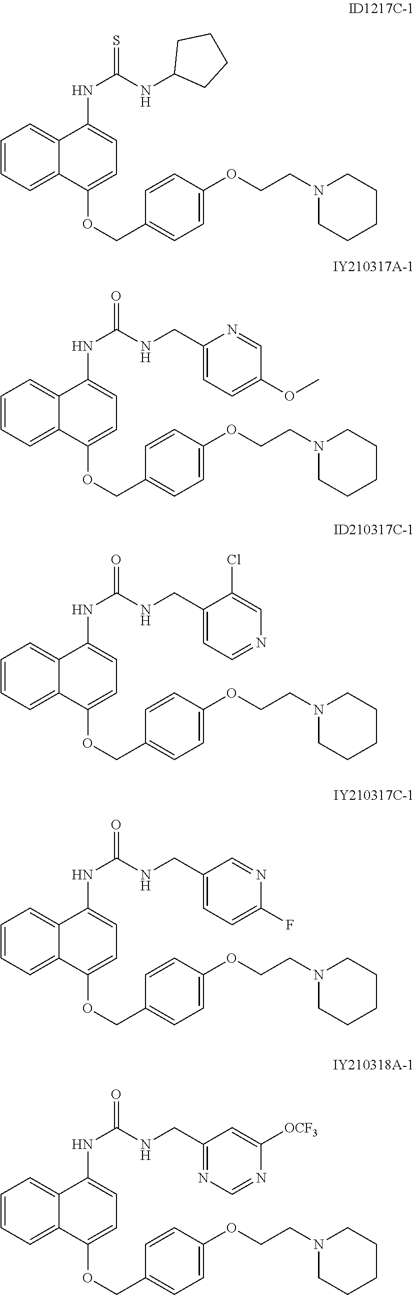 [SMILES: S=C(Nc1ccc(OCc2ccc(OCCN3CCCCC3)cc2)c2ccccc12)NC1CCCC1 ; COc1ccc(CNC(=O)Nc2ccc(OCc3ccc(OCCN4CCCCC4)cc3)c3ccccc23)nc1 ; O=C(NCc1ccncc1Cl)Nc1ccc(OCc2ccc(OCCN3CCCCC3)cc2)c2ccccc12 ; O=C(NCc1ccc(F)nc1)Nc1ccc(OCc2ccc(OCCN3CCCCC3)cc2)c2ccccc12 ; O=C(NCc1cc(OC(F)(F)F)ncn1)Nc1ccc(OCc2ccc(OCCN3CCCCC3)cc2)c2ccccc12]

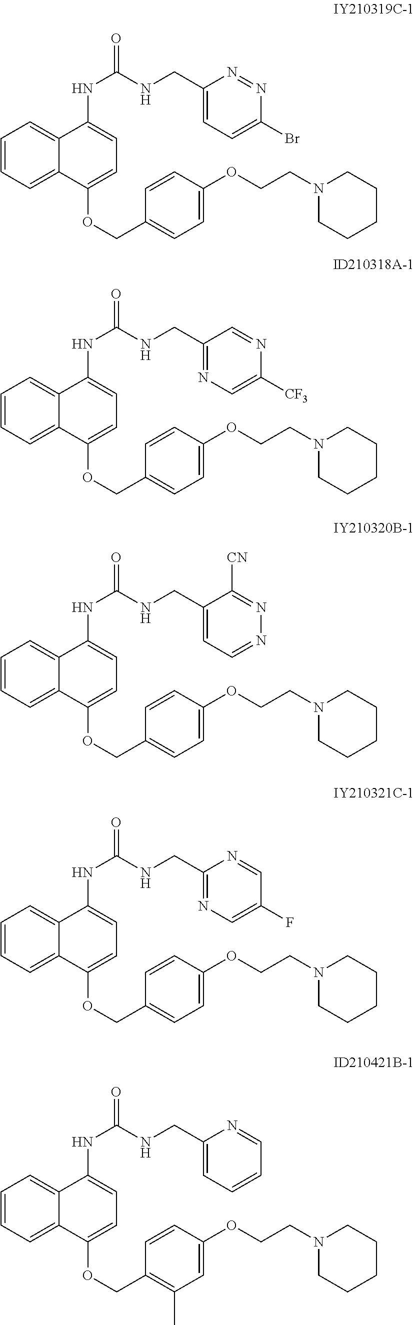 [SMILES: O=C(NCc1ccc(Br)nn1)Nc1ccc(OCc2ccc(OCCN3CCCCC3)cc2)c2ccccc12 ; O=C(NCc1cnc(C(F)(F)F)cn1)Nc1ccc(OCc2ccc(OCCN3CCCCC3)cc2)c2ccccc12 ; N#Cc1nnccc1CNC(=O)Nc1ccc(OCc2ccc(OCCN3CCCCC3)cc2)c2ccccc12 ; O=C(NCc1ncc(F)cn1)Nc1ccc(OCc2ccc(OCCN3CCCCC3)cc2)c2ccccc12 ; Cc1cc(OCCN2CCCCC2)ccc1COc1ccc(NC(=O)NCc2ccccn2)c2ccccc12]

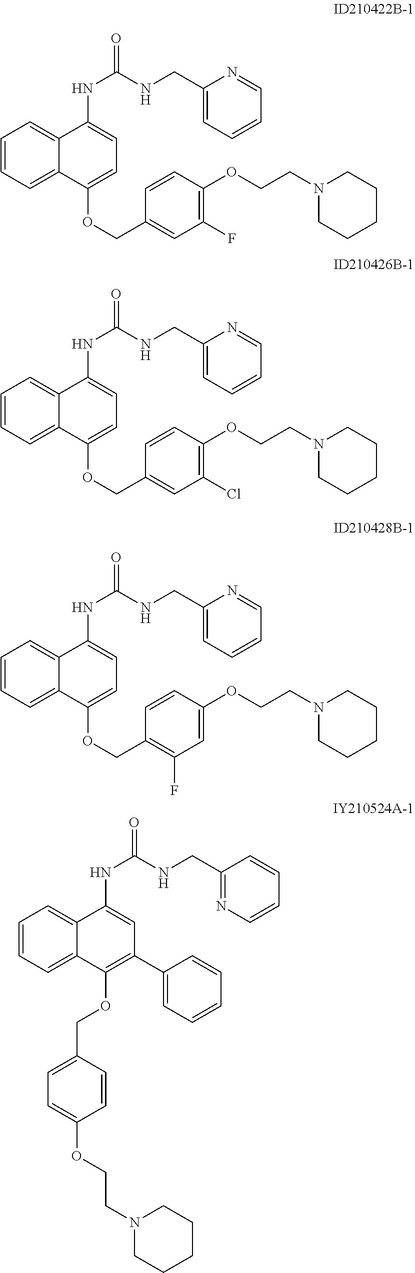 [SMILES: O=C(NCc1ccccn1)Nc1ccc(OCc2ccc(OCCN3CCCCC3)c(F)c2)c2ccccc12 ; O=C(NCc1ccccn1)Nc1ccc(OCc2ccc(OCCN3CCCCC3)c(Cl)c2)c2ccccc12 ; O=C(NCc1ccccn1)Nc1ccc(OCc2ccc(OCCN3CCCCC3)cc2F)c2ccccc12 ; O=C(NCc1ccccn1)Nc1cc(-c2ccccc2)c(OCc2ccc(OCCN3CCCCC3)cc2)c2ccccc12]

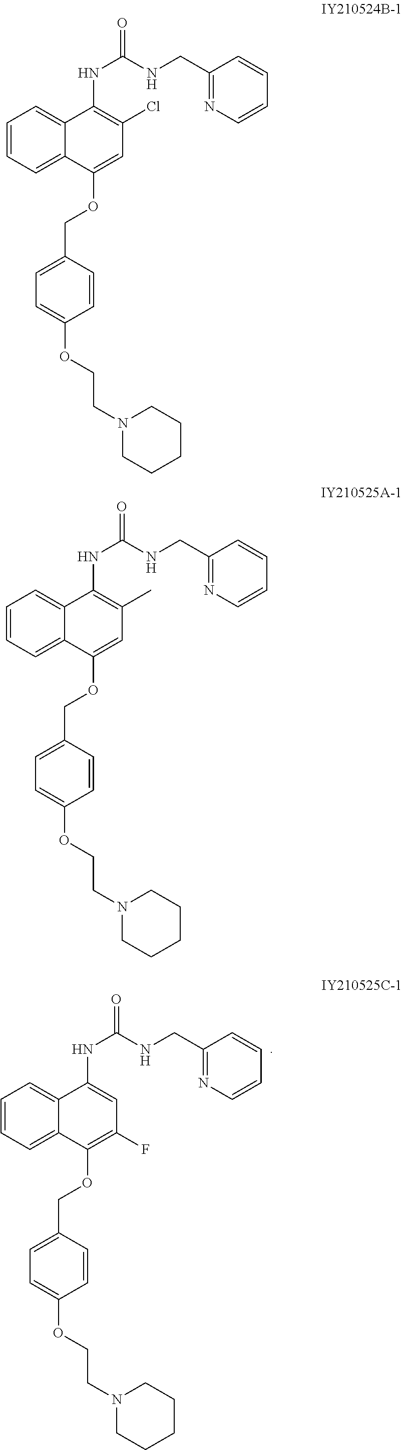

3. A biologically acceptable salt, being formed by contacting the compound of claim 1 with at least an acid selected from the group consisting of acetic acid, dihydrofolic acid, benzoic acid, citric acid, sorbic acid, propionic acid, oxalic acid, fumaric acid, maleic acid, hydrochloric acid, malic acid, phosphoric acid, sulfite, sulfuric acid, vanillic acid, tartaric acid, ascorbic acid, boric acid, lactic acid, and ethylenediaminetetraacetic acid.

4. A method for preparing the compound of claim 1, comprising:
1) dissolving 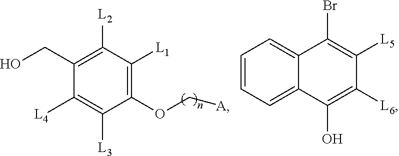
and triphenylphosphine in tetrahydrofuran to yield a mixture, adding diisopropyl azodicarboxylate at −5-5° C. to the mixture, and stirring at room temperature, to yield 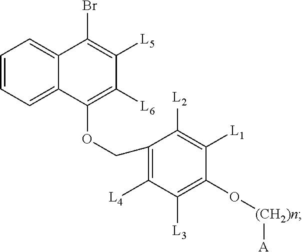
2) dissolving 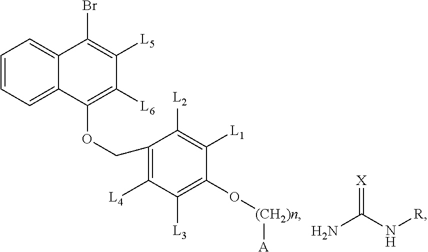
and potassium tert-butoxide in methylbenzene, and adding Pd₂(dba)₃ and 4,5-bis(diphenylphosphino)-9,9-dimethylxanthene at nitrogen atmosphere to the methylbenzene, allowing to react at 110° C., to yield

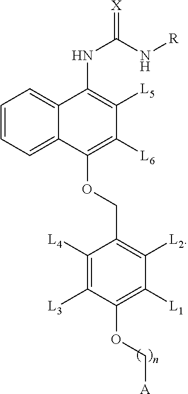

5. The method of claim 4, wherein 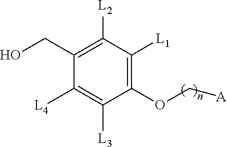
is prepared as follows:
a) dissolving 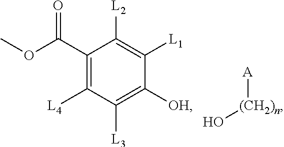
and triphenylphosphine in tetrahydrofuran, and adding diisopropyl azodicarboxylate to a resulting mixture at −5-5° C. under protective atmosphere, and stirring at room temperature, to yield 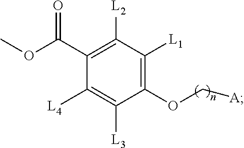
and
b) dissolving 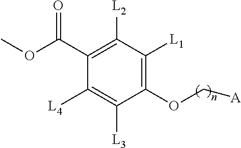
in tetrahydrofuran, adding lithium aluminum hydride in batches at −5-5C, and stirring at room temperature, to yield

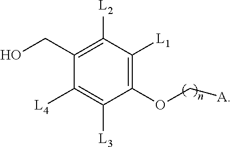

6. The method of claim 4, wherein
in 1), a molar ratio of 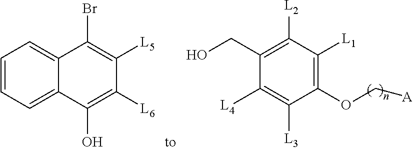
to triphenylphosphine to diisopropyl azodicarboxylate is 1:1:1.2:1.2;
in 2), a molar ratio of 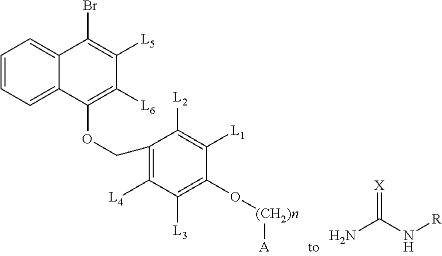
to potassium tert-butoxide to 4,5-bis(diphenylphosphino)-9,9-dimethylxanthene is 1:1:1.3:0.05:0.1.

7. The method of claim 5, wherein in a), a molar ratio of 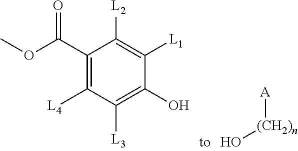
to triphenylphosphine to diisopropyl azodicarboxylate is 1:1.2:1.2:1.2; and in b), a molar ratio of 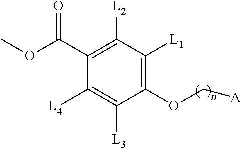
to lithium aluminum hydride is 1:1.

8. A method for treating a tumor comprising administering a patient in need thereof a naphthylurea compound of claim 1 or a biologically acceptable salt thereof, the tumor being a JAKs or STAT3 signaling-related disease.

9. The method of claim 8, wherein the tumor is liver cancer, breast cancer, lung cancer, or leukemia.
